(19) United States
(12) Patent Application Publication
(10) Pub. No.: US 2020/0325520 A1
(43) Pub. Date: Oct. 15, 2020

(54) NUCLEIC ACID QUANTIFICATION METHOD USING STABLE ISOTOPE-LABELLED NUCLEIC ACID AS INTERNAL STANDARD AND USE OF THE SAME

(71) Applicant: KOREA RESEARCH INSTITUTE OF STANDARDS AND SCIENCE, Daejeon (KR)

(72) Inventors: In Chul YANG, Daejeon (KR); Ha Jeong KWON, Daejeon (KR); Ji Seon JEONG, Daejeon (KR); Young Kyung BAE, Daejeon (KR)

(73) Assignee: KOREA RESEARCH INSTITUTE OF STANDARDS AND SCIENCE, Daejeon (KR)

(21) Appl. No.: 16/760,296

(22) PCT Filed: Oct. 30, 2018
(86) PCT No.: PCT/KR2018/013028

§ 371 Date: Apr. 29, 2020

(30) Foreign Application Priority Data
Oct. 30, 2017 (KR) 10-2017-0142509

(51) Int. Cl. C12Q 1/68; C12Q 1/6806

(52) U.S. Cl. CPC C12Q 1/68; G01N 30/7233; C12Q 1/6806

(57) ABSTRACT

In order to quantitatively analyze nucleic acids present in a sample or a complex medium, a nucleic acid extraction or purification process is required. However, the yield of nucleic acid extraction and purification is greatly variable depending on the purification principle and the characteristics of kit and sample used. Hence, efficient normalization of nucleic acid extraction and purification yield is a prerequisite for accurate quantitative analysis of nucleic acid based on the original sample. The present invention relates to a quantitative analysis method of a nucleic acid present in a sample or a complex medium without amplification of a target nucleic acid.

### TECHNICAL FIELD

The present invention relates to a quantitative analysis method of nucleic acids (DNA and RNA) with increased accuracy and reliability. Specifically, the present invention relates to a quantitative analysis method of nucleic acids using stable isotope-labelled nucleic acids (DNA or RNA) (hereinafter referred to as ‘SILD’) as an internal standard.

### BACKGROUND ART

Gene analysis includes the process of amplifying a specific gene and analyzing its sequencing by PCR and sequencing technology. Gene analysis is widely utilized in medical fields such as disease diagnosis, mutation detection, and detection of pathogenic bacteria and viruses; food and hygiene fields such as detection of genetically modified agricultural products, identification of origin of food materials, and detection of microorganisms contaminating food materials; environmental fields such as microbial community analysis, analysis of toxicity to organisms, and conservation of biodiversity; and forensic medicine fields such as paternity, personal identification, and suspect identification.

By the development of next generation sequencing (NGS) technology, it is possible to simultaneously analyze dozens or hundreds of different samples or to simultaneously analyze thousands and tens of thousands of genomes at a high efficiency. NGS technology is extremely usefully utilized in various fields such as analysis of expression patterns of all genes by transcriptome analysis and analysis of large-scale and high-precision microbial communities; finding of population genetic characteristics and disease markers by cohort analysis; and disease prediction and personalized medicine by personal genome analysis.

Recently, ‘circulating cell free nucleic acids’ have been discovered in blood. Subsequent studies have revealed that circulating nucleic acids are of great medical importance, and there is a great need for a precise analysis method to use these.

A phenomenon has been found that the amount of circulating nucleic acids in the blood is 20 to 100 ng/ml in the normal state but it greatly increases to 200 to 500 ng/ml when cancer such as breast cancer and blood cancer onsets. It has been reported that the amount of circulating nucleic acids in the blood changes not only at the time of onset of cancer but also at the time of myocardial infarction, infection, acute inflammation, excessive exercise, and stress. In other words, the early diagnosis of major diseases is possible by simply measuring the amount of circulating nucleic acids in the blood. Above all, accurate and reliable quantitative analysis of circulating nucleic acids in the blood is required for this.

As nucleic acid quantification methods, UV spectrometry, quantitative nucleic acid amplification (qPCR), digital nucleic acid amplification (digital PCR), fluorescence quantification and the like are often used. By these quantification methods, it is not possible to accurately quantify nucleic acids without a purification process since the analysis is disturbed by other components present in the sample or medium. However, a quantitative analysis method of nucleic acids in a complex medium with high accuracy and reliability has not been proposed.

Patent Literature 1 relates to a method for quantifying nucleic acids, which includes adding different amounts of each of various nucleic acid constructs which can be distinguished from an analyte nucleic acid in a sample and can be simultaneously amplified with this nucleic acid to the sample; treating the sample through a nucleic acid amplification procedure using an amplification reagent capable of reacting with the analyte nucleic acid and the nucleic acid constructs; and calculating an amount of the analyte nucleic acid from the relative amounts. The respective nucleic acid constructs are different from each other; and the nucleic acid constructs can be distinguished from each other and from the analyte nucleic acid. The nucleic acid construct is similar to the nucleic acid construct and the analyte nucleic acid in that it can react with the same amplification reagent.

However, the above method cannot be regarded as a reliable quantitative analysis method in that the various nucleic acid constructs used in the creation of the calibration curve may continue to vary depending on the nucleic acid to be analyzed and there is a premise that the nucleic acid construct is required to be amplified at the same rate as the analyte.

Patent Literature 2 relates to the use of a universal reference nucleic acid to create a calibration curve from which the characteristic level of a target nucleic acid in a sample can be calculated. Patent Literature 2 relates to a quantification method by introduction of a universal reference nucleic acid labelled with a known amount of a fluorophore. As in Patent Literature 1, an error is basically inherent in this method as well since there is a premise that the nucleic acid used in the creation of calibration curve is also required to be amplified at the same rate.

As described above, a method capable of precisely quantifying a nucleic acid without amplification of target nucleic acid has not been so far proposed.

### CITATION LIST

### Patent Literature

[Patent Literature 1] Korean Patent Registration No. 10-0312800

[Patent Literature 2] Korean Patent Publication No. 10-2017-0083053

### SUMMARY OF INVENTION

### Technical Problem

In order to quantitatively analyze nucleic acids present in a sample or a complex medium, a nucleic acid extraction or purification process is required. However, the yield of nucleic acid extraction and purification is greatly variable depending on the purification principle and the characteristics of kit and sample used. Hence, efficient normalization of nucleic acid extraction and purification yield is a prerequisite for accurate quantitative analysis of nucleic acid based on the original sample. The present invention is intended to provide a quantitative analysis method of a nucleic acid present in a sample or a complex medium without amplification of target nucleic acid.

### Solution to Problem

The present invention relates to a quantitative analysis method of a nucleic acid using SILD as an internal standard as a method for increasing the accuracy and reliability of quantitative analysis of nucleic acids (DNA and RNA) in a sample.

In order to accurately quantitatively analyze nucleic acids present in a sample or a complex medium, a nucleic acid extraction or purification process is required. However, the yield of nucleic acid extraction and purification is greatly variable depending on the purification principle and the characteristics of kit and sample used. Hence, efficient normalization of nucleic acid extraction and purification yield is a prerequisite for accurate quantitative analysis of nucleic acid based on the original sample.

The present invention relates to a method in which SILD is used as an internal standard to normalize the yield in the nucleic acid purification and pretreatment process.

As a method often utilized to indirectly normalize the yield of nucleic acid extraction and purification, a method is also used in which the yield of extraction and purification is calculated by adding a known amount of a specific gene as an internal standard to the sample and then measuring the amount of this gene again after extraction and purification. However, the nucleic acid to be added as an internal standard has a single size while the sizes of purification target nucleic acids vary, and the nucleic acid extraction efficiency is also greatly affected by the size of nucleic acid, thus this method has a drawback of being hardly utilized for normalization of the overall extraction efficiency. In addition, in order to solve this size problem, there is also a method in which the yield depending on the kit, the experimenter and the like is predicted by conducting separate extraction and purification reactions and quantitative analysis using known amounts of nucleic acids having the same size distribution as the purification target nucleic acids as external standards. However, it is difficult to say that this method is a perfect method for efficiency normalization since the yield of nucleic acid purification may be fundamentally different for each reaction vessel and is also different depending on the form of medium in the sample. Accordingly, in order to overcome these drawbacks of the existing methods, the present invention has been achieved by inventing a method for normalizing the purification efficiency of the entire nucleic acids while using an internal standard.

In the present invention, SILD is used as an internal standard as a method for normalizing the yield of nucleic acid extraction and purification. SILD has the same chemical and biological properties as an analyte normal nucleic acid, but the molecular weight thereof is different from that of the normal nucleic acid by the presence of stable isotopes (¹³C, ¹⁵N). Moreover, this difference in molecular weight makes it possible to detect and quantify SILD and the normal nucleic acid as different charge-to-mass ratios (m/z) in a mass spectrometer, which is a final analytical instrument. In other words, the amount of the normal nucleic acid calculated from each sample and the amount of the nucleic acid as an internal standard can be simultaneously and separately quantified. Moreover, the properties of the internal standard calculated from the sample are the same as the efficiency of extraction, purification, enzymatic reaction, and mass spectrometric analysis of the analyte nucleic acid, and thus the signal value of the internal standard is a measure of the reaction efficiency of the analyte for the same reaction.

If the amount of nucleic acid added as an internal standard is already known or the internal standard is added to a standard for nucleic acid quantification in the same amount, this analysis method is commonly called isotope dilution mass spectrometry and is a method used in quantitative analysis of substances in the field of analytical chemistry. However, for isotope dilution mass spectrometry, it is required to prepare an internal standard substituted with an isotope for the analyte. Substances mainly targeted in the field of analytical chemistry have a small molecular weight and a simple structure, thus it is relatively easy to prepare an internal standard substituted with an isotope, and the internal standard can be purchased from a commercial reagent company. However, in the case of nucleic acids, the molecular weight thereof is significantly great (genomic nucleic acids usually have a length of 10 kb and a molecular weight of 7 MDa or more and circulating nucleic acids in the blood have a length of 150 bp and a molecular weight of about 100 kDa) and the structure thereof is complicated, and thus it is not easy to prepare an isotope dilution internal standard. Despite these difficulties, a method for labelling the entire nucleic acids with stable isotopes has been developed by cultivating Escherichia coli in a medium containing only inorganic elements and a medium additionally containing a nitrogen source (ammonium sulfate) and a carbon source (glycerol) which are substituted with isotopes. In the present invention, an Escherichia coli genomic nucleic acid labelled with a stable isotope is produced by focusing on this technology, and this nucleic acid is used as an internal standard for the analysis of nucleic acids in a medium.

SILD, which is used as an internal standard, is added to the analyte sample and the comparison target sample (control or standard) in the same amount at the start of analysis. SILD substituted with stable isotopes such as ¹³C and ¹⁵N have chemically identical properties to the analyte nucleic acid contained in the original sample and thus has the same efficiency in principle not only in the extraction and purification process of nucleic acid but also in subsequent enzymatic reaction, mass spectrometric analysis process and the like. In addition, the signal value of the added nucleic acid as the internal standard can be separated since a charge-to-mass (m/z) value different from that of the analyte nucleic acid is detected in the mass spectrometric analysis as the final analysis step because of the substitution with stable isotopes.

Liquid chromatography-mass spectrometry (LC-MS) is used in the mass spectrometric analysis process.

Meanwhile, at the start of analysis, SILD is added to the analyte sample and the comparison target sample (control or standard) in the same amount, and thus the instrument signal value (peak area by mass spectrometer) of the internal standard derived from each sample is an objective measure of the efficiency of purification, enzymatic reaction, and mass spectrometric analysis for each sample.

The present invention is a quantitative analysis method of a nucleic acid comprising: 1) preparing a nucleic acid (SILD) substituted with stable isotopes of ¹³C and/or ¹⁵N; 2) adding the substituted nucleic acid (SILD) as an internal standard to an analyte sample and a control sample in the same amount; 3) obtaining a nucleic acid from the analyte sample and a nucleic acid from the control sample; 4) hydrolyzing the nucleic acids obtained in the step 3) to a single nucleoside level; 5) attaining detection values of a normal nucleoside and a nucleoside derived from the substituted nucleic acid (SILD) from the nucleosides obtained in the step 4) in mass spectrometric analysis; and 6) normalizing an amount of the nucleic acid in the analyte sample by utilizing a characteristic that the detection value of the nucleoside derived from the substituted nucleic acid (SILD) is the same in the analyte sample and the control sample.

Here, the nucleic acid is DNA or RNA, and the sample is at least one or more of whole blood, plasma, serum, urine, saliva, sweat, milk, animal extract, plant extract, cell extract, cell culture, drinking water, service water, sewage, river water, or seawater.

SILD substituted with ¹³C and/or ¹⁵N is derived from one of Escherichia coli, a human, a mouse, yeast, a plant, a fruit fly, or Caenorhabditis elegans and is preferably derived from Escherichia coli.

The step of obtaining a nucleic acid from the sample may be extraction and purification.

The method for hydrolyzing the nucleic acid to a single nucleoside level is at least one or more of an enzymatic reaction, an acid treatment, a heat treatment, a radiation treatment, or an ultrasonic treatment. In particular, by the hydrolyzation, 99.5% (by weight) or more of the entire nucleic acid is hydrolyzed to a single nucleoside.

The normalization step is calculation by the following equation.

Nucleic acid (analyte)=detection value of nucleic acid (analyte)×nucleic acid (control)/detection value of nucleic acid (control)×detection value of SILD (control)/detection value of SILD (analyte)

Here, the nucleic acid (analyte) denotes an amount of a nucleic acid in an analyte sample, the detection value of nucleic acid (analyte) denotes a detection value of a nucleic acid in an analyte sample in mass spectrometric analysis, the nucleic acid (control) denotes an amount of a nucleic acid in a control sample, the detection value of nucleic acid (control) denotes a detection value of a nucleic acid in a control sample in mass spectrometric analysis, the detection value of SILD (control) denotes a detection value of a substituted nucleic acid (SILD) in a control sample in mass spectrometric analysis, and the detection value of SILD (analyte) denotes a detection value of a substituted nucleic acid (SILD) in an analyte sample in mass spectrometric analysis.

### Advantageous Effects of Invention

The effect of the present invention is to make it possible to normalize the difference in yield occurring in the extraction and purification process of nucleic acids in a medium by using SILD as an internal standard. In addition, the added internal standard also normalizes the efficiency of enzymatic reaction after purification and mass spectrometric analysis. For example, when some impurities remain, the efficiency of enzymatic reaction or the ionization efficiency in mass spectrometric analysis may change, and the interference effect received by the analyte can be normalized using the signal value ratio of the internal standard since the internal standard also receives this effect to the same extent. Overall, the use of SILD as an internal standard makes it possible to improve the accuracy of quantitative analysis of nucleic acids in a medium by normalizing the efficiency of all the procedures and reactions conducted to quantitatively analyze nucleic acids in a medium sample.

### BRIEF DESCRIPTION OF DRAWINGS

FIG. 1 is a schematic diagram illustrating a process for quantifying a nucleic acid in a medium by using SILD as an internal standard. ‘⋆’ denotes a stable isotope-labelled substance.

FIG. 2 is the mass spectrometric analysis results for Escherichia coli genomic DNA produced to use SILD as an internal standard.

FIG. 3 is a diagram illustrating the results attained by normalizing the DNA extraction and purification efficiency by using SILD as an internal standard.

FIG. 4 is the results attained by measuring the amount of free nucleic acids (cell free DNA) in human serum by using SILD as an internal standard.

### DESCRIPTION OF EMBODIMENTS

The present invention is a quantification method of a nucleic acid in a medium including 1) adding SILD as an internal standard to an analyte sample and a comparison target sample (control or standard) in the same amount, 2) extracting or purifying a nucleic acid from each sample, 3) hydrolyzing the purified nucleic acid to a single nucleoside level through an enzymatic reaction, 4) separating, detecting, and quantifying each nucleoside and a stable isotope-substituted nucleoside by liquid chromatography-mass spectrometry (LC-MS), and 5) normalizing a difference in efficiency of the whole steps by utilizing a signal value of the internal standard and quantitatively calculating an amount of the nucleic acid in the analyte sample.

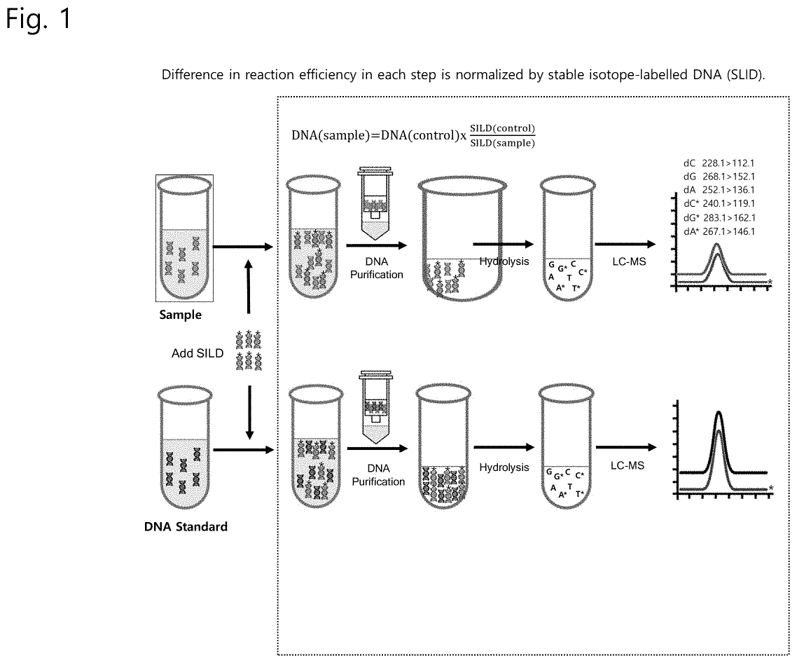

FIG. 1 is a schematic diagram illustrating a process for quantifying a nucleic acid in a medium by using SILD as an internal standard. SILD is added to the analyte sample and the comparison target sample (or standard sample) in the same amount, and then the two samples are sequentially subjected to extraction and purification, hydrolysis by enzymatic reaction, and mass spectrometric analysis. Finally, the signal value of SILD which has been added to the two samples in the same amount in the mass spectrometric analysis results is a measure of the overall reaction efficiency and yield for the two samples. The absolute or relative amount of a nucleic acid in a medium can be calculated by the formula depicted in the figure, exactly the equation described below.

Nucleic acid (analyte)=detection value of nucleic acid (analyte)×nucleic acid (control)/detection value of nucleic acid (control)×detection value of SILD (control)/detection value of SILD (analyte)

Here, the nucleic acid (analyte) denotes the amount of nucleic acid in the analyte sample, the detection value of nucleic acid (analyte) denotes the detection value of nucleic acid in the analyte sample in mass spectrometric analysis, the nucleic acid (control) denotes the amount of nucleic acid in the control sample, the detection value of nucleic acid (control) denotes the detection value of nucleic acid in the control sample in mass spectrometric analysis, the detection value of SILD (control) denotes the detection value of substituted nucleic acid (SILD) in the control sample in mass spectrometric analysis, and the detection value of SILD (analyte) denotes the detection value of substituted nucleic acid (SILD) in the analyte sample in mass spectrometric analysis.

In order to implement the present invention, it is first required to produce SILD.

1. Production and Verification of SILD

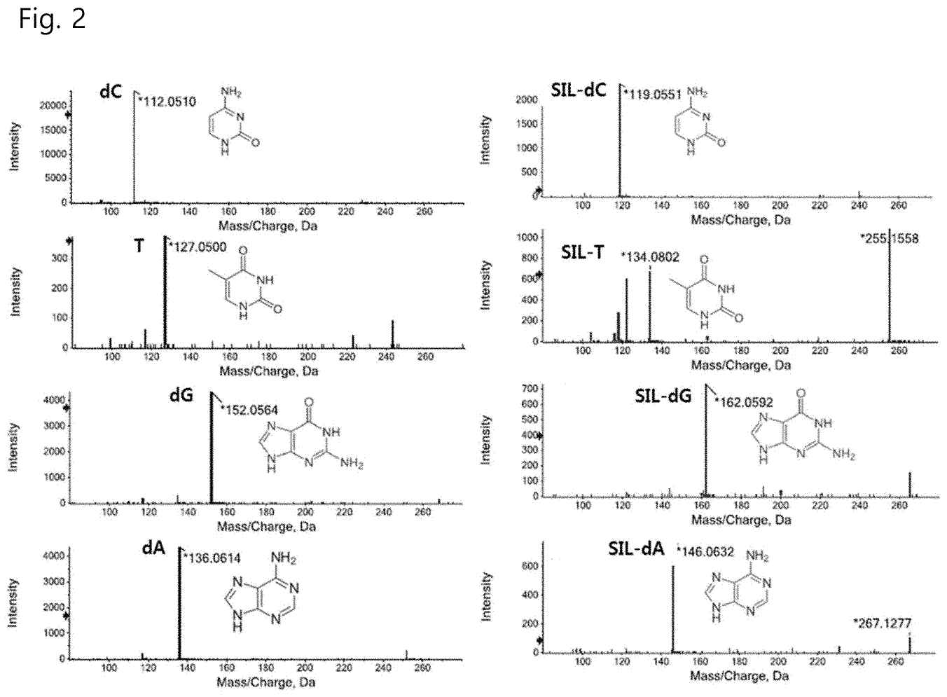

The production of SILD was conducted according to the method described in the reference (Appl Microbiol Biotechnol (2010) 88: 771-779). Briefly, (NH₄)₂SO₄ substituted with ¹⁵N was used in the composition of the LMR medium (176 mM KH₂PO₄, 25 mM NaOH, 10 μl H₂SO₄, 12.6 mM (NH₄)₂SO₄, 2 mM MgSO₄, 10 micromole FeSO₄, 0.2% trace metal solution) composed only of essential inorganic elements (Cambridge Isotope Laboratory), and a medium to which glycerol substituted with 0.2% of ¹³C as a carbon source was used. As Escherichia coli, a standard strain KCTC11 was used. Genomic DNA extraction from Escherichia coli cultured in stable isotope medium was conducted using Genelute Bacterial genomic DNA kit (Sigma-Aldrich). In order to verify that the extracted genomic DNA is favorably labelled with stable isotopes, about 500 ng of DNA was hydrolyzed to a nucleoside (dNMP) level using DNase I (Takara) and Phosphodiesterase I (Affymetrics) and each nucleoside was detected using LC-Quadrupole-TOF (AB SCIEX 5600) mass spectrometer (see FIG. 2).

As can be seen from FIG. 2, the difference in molecular weight between Escherichia coli DNA cultured in a normal medium and Escherichia coli DNA cultured in a stable isotope medium is 12 in the case of dCMP and TMP and is 15 in the case of dAMP and dGMP. This difference corresponds to the difference based on the assumption that both carbon and nitrogen in each nucleoside are substituted. In addition, normal nucleosides having a small molecular weight are not detected in DNA cultured in a stable isotope medium. Hence, according to the method implemented in the present invention, it has been verified that Escherichia coli DNA is labelled with stable isotopes at a level close to 100%.

2. Normalization of Extraction and Purification Efficiency of DNA in Medium and Quantitative Analysis of DNA

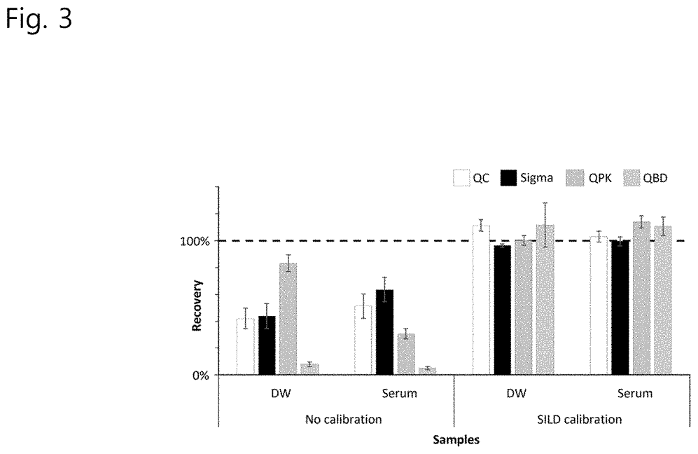

In order to verify that the extraction and purification efficiency of DNA in a medium is properly normalized when stable isotope-labelled Escherichia coli DNA is added as an internal standard, a buffer (hGH buffer: 2.25% mannitol, 0.5% glycine, 0.15% sodium phosphate, 5 mg/mL bovine serum albumin) for protein drug storage was selected as a representative medium. DNA as an analyte sample was added to the hGH buffer in a known amount of 100 ng and SILD as an internal standard was added to the hGH buffer in an amount corresponding to about 100 ng. The same amount of SILD was also added to human placental DNA and dNMP samples with values already known as standards for quantitation. The SILD-added samples were extracted and purified using four different kinds of kits of PCR purification kit (QPK, Qiagen), QiaAmp DNA Blood mini kit (QBD, Qiagen), Serum/plasma cell free DNA midi kit (Sigma, Sigma-Aldrich), and QiaAmp circulating nucleic acid kit (QC, Qiagen) (FIG. 3). DNA was hydrolyzed to a nucleotide (dNMP) level using DNase I (Takara) and Phosphodiesterase I (Affymetrics) and further hydrolyzed to nucleoside (dN) using Shrimp alkaline phosphatase (Takara). The four kinds of hydrolyzed nucleosides were quantitatively analyzed using LC-Quadrupole-TOF (AB SCIEX 5600) mass spectrometer. The amount of DNA in the medium was calculated by applying the following equation based on the peak areas of normal nucleosides and SILD-derived nucleosides calculated from each purified sample. The following equation can be applied only when the analyte sample and the internal standard were used in the same amount of 100 ng.

DNA (sample)=(DNA (standard)×SILD (standard)/SILD (sample)

The quantification results for nucleic acid before and after the normalization for every kit are compared with each other in FIG. 3. The quantification results attained without normalization using SILD show quantitative values to be 20% to 70% of the initial reference values depending on the kit. On the other hand, quantitative values to be 90% to 105% of the reference values were attained in the results attained by conducting normalization of purification and hydrolysis reaction using SILD proposed in the present invention. These results indicate that the accuracy of quantification can be dramatically increased by using a SILD internal standard in the quantitative analysis of nucleic acids in a medium. In addition, it has been verified that the use of SILD internal standard enables highly accurate nucleic acid quantification even when the nucleic acid purification process is omitted.

As can be seen from FIG. 3, the peak area of nucleoside is about 50% of the reference value when normalization is not conducted, and thus it can be seen that the efficiency of enzymatic hydrolysis and the ionization efficiency in mass spectrometric analysis are lower than those in the case of purified nucleic acids. It is interpreted that the efficiency is decreased because the impediments contained in the hGH buffer have not been removed by purification. However, when SILD is used, even such low hydrolysis and ionization efficiencies are normalized, thus the final nucleic acid quantification value is 101.5% of the reference value, and significantly accurate quantification is possible. Based on these observation results, it can be concluded that the amount of nucleic acid in a medium can be measured significantly accurately regardless of the kind of extraction and purification kit and further even when purification is not conducted when SILD is used as an internal standard.

3. Quantitative Analysis of Free DNA in Human Serum

In order to verify that the extraction and purification efficiency of DNA in a medium is properly normalized when stable isotope-labelled Escherichia coli DNA as an internal standard is added to the medium, free DNA in human serum was quantified. About 50 ng of SILD was added to 0.5 ml of each of 16 human serum samples prior to the purification of free DNA in serum. In addition, SILD as an internal standard was also added to a dNMP standard mixture with a known amount (four individual nucleotides each having a concentration of 20 ng/mL) as a reference for quantification in the same amount as the above.

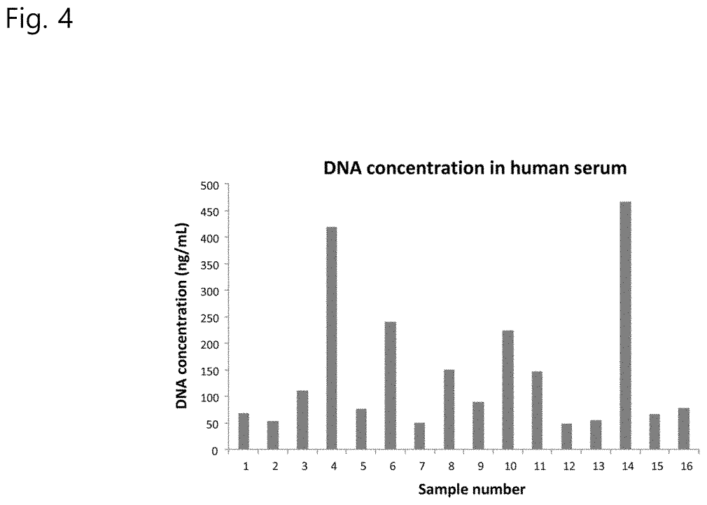

The SILD-added samples were subjected to DNA extraction using Circulating cell free DNA purification kit (Qiagen). The extracted DNA was subjected to hydrolysis in the same manner as described above and then quantitatively analyzed by LC-MS. The results are illustrated in FIG. 4. The range of the measured values illustrated in FIG. 4 is 50 to 500 ng/ml, and these values are significantly higher than 20 to 100 ng/ml generally calculated in experiments in which DNA extraction and purification efficiency is not normalized. Considering that the purification efficiency of the kit used for DNA purification is about 40%, it is judged that a measured value as high as about two times is attained since the measurement method used in the present invention completely normalizes the purification efficiency. Through the results described above, it has been verified that the ‘measurement method of DNA in a medium using a stable isotope-labelled DNA as an internal standard’, which is proposed in the present invention is a method which enables accurate quantification by collectively normalizing the purification efficiency of DNA, the efficiency of enzymatic hydrolysis, and the variability in LC-MS.

### INDUSTRIAL APPLICABILITY

The present invention is to normalize the difference in yield occurring in the extraction and purification process of a nucleic acid in a medium by using SILD as an internal standard. In addition, the added internal standard also normalizes the efficiency of enzymatic reaction after purification and mass spectrometric analysis. For example, when some impurities remain, the efficiency of enzymatic reaction or the ionization efficiency in mass spectrometric analysis may change, and the interference effect received by the analyte can be normalized using the signal value ratio of the internal standard since the internal standard also receives this effect to the same extent. Overall, the use of SILD as an internal standard makes it possible to improve the accuracy of quantitative analysis of nucleic acids in a medium by normalizing the efficiency of all the procedures and reactions conducted to quantitatively analyze nucleic acids in a medium sample.

1. A quantitative analysis method of a nucleic acid comprising:
1) preparing a nucleic acid (SILD) substituted with stable isotopes of ¹³C and/or ¹⁵N;
2) adding the substituted nucleic acid (SILD) as an internal standard to an analyte sample and a control sample in the same amount;
3) obtaining a nucleic acid from the analyte sample and a nucleic acid from the control sample;
4) hydrolyzing the nucleic acids obtained in the step 3) to a single nucleoside level;
5) attaining detection values of a normal nucleoside and a nucleoside derived from the substituted nucleic acid (SILD) from the nucleosides obtained in the step 4) in mass spectrometric analysis; and
6) normalizing an amount of the nucleic acid in the analyte sample by utilizing a characteristic that the detection value of the nucleoside derived from the substituted nucleic acid (SILD) is the same in the analyte sample and the control sample.

2. The quantitative analysis method of a nucleic acid according to claim 1, wherein
the nucleic acid is DNA or RNA, and
the sample is at least one or more of whole blood, plasma, serum, urine, saliva, sweat, milk, animal extract, plant extract, cell extract, cell culture, drinking water, service water, sewage, river water, or seawater.

3. The quantitative analysis method of a nucleic acid according to claim 2, wherein the nucleic acid is DNA and the sample is serum.

4. The quantitative analysis method of a nucleic acid according to claim 1, wherein the substituted nucleic acid (SILD) is derived from one of Escherichia coli, a human, a mouse, yeast, a plant, a fruit fly, or Caenorhabditis elegans.

5. The quantitative analysis method of a nucleic acid according to claim 4, wherein the substituted nucleic acid (SILD) is derived from Escherichia coli.

6. The quantitative analysis method of a nucleic acid according to claim 1, wherein the obtaining step is extraction and purification.

7. The quantitative analysis method of a nucleic acid according to claim 1, wherein the hydrolysis is at least one or more of an enzymatic reaction, an acid treatment, a heat treatment, a radiation treatment, or an ultrasonic treatment.

8. The quantitative analysis method of a nucleic acid according to claim 1, wherein the single nucleoside level is that 99.5% (by weight) or more of an entire nucleic acid is hydrolyzed to a single nucleoside.

9. The quantitative analysis method of a nucleic acid according to claim 1, wherein the normalization step is calculation by the following equation:
nucleic acid (analyte)=detection value of nucleic acid (analyte)×nucleic acid (control)/detection value of nucleic acid (control)×detection value of SILD (control)/detection value of SILD (analyte)
where the nucleic acid (analyte) denotes an amount of a nucleic acid in an analyte sample, the detection value of nucleic acid (analyte) denotes a detection value of a nucleic acid in an analyte sample in mass spectrometric analysis, the nucleic acid (control) denotes an amount of a nucleic acid in a control sample, the detection value of nucleic acid (control) denotes a detection value of a nucleic acid in a control sample in mass spectrometric analysis, the detection value of SILD (control) denotes a detection value of a substituted nucleic acid (SILD) in a control sample in mass spectrometric analysis, and the detection value of SILD (analyte) denotes a detection value of a substituted nucleic acid (SILD) in an analyte sample in mass spectrometric analysis.
